(19) United States
(12) Patent Application Publication
(10) Pub. No.: US 2003/0180720 A1
(43) Pub. Date: Sep. 25, 2003

(54) Analyte-shaped cavities associated with enhancing particle structures for analyte detection

(76) Inventors: David I. Kreimer, Berkeley, CA (US); Oleg A. Yevin, Oakland, CA (US); Thomas H. Nufert, Walnut Creek, CA (US)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/364,160

(22) Filed: Feb. 11, 2003

Related U.S. Application Data
(63) Continuation-in-part of application No. 10/298,725, filed on Nov. 18, 2002.
(63) Continuation-in-part of application No. 10/294,385, filed on Nov. 14, 2002.
(63) Continuation-in-part of application No. 09/925,189, filed on Aug. 8, 2001.
(63) Continuation-in-part of application No. 09/815,909, filed on Mar. 23, 2001.
(63) Continuation-in-part of application No. 09/670,453, filed on Sep. 26, 2000.
(60) Provisional application No. 60/356,254, filed on Feb. 11, 2002.
(60) Provisional application No. 60/156,195, filed on Sep. 27, 1999.
(60) Provisional application No. 60/156,471, filed on Sep. 27, 1999.
(60) Provisional application No. 60/156,145, filed on Sep. 27, 1999.

(51) Int. Cl. C12Q 1/70; C12Q 1/68; C12M 1/34; G01N 33/543

(52) U.S. Cl. 435/5; 436/518; 435/287.2

(57) ABSTRACT

This invention comprises novel matrices comprising enhancing structures associated with analyte-shaped cavities. The structures are useful for Raman spectroscopic analyses of analytes in complex solutions. Analytes that can be detected using these methods include nucleic acids, proteins, and other molecules or viruses that can specifically bind to the arrays. Enhancing structures are disclosed that enhance the Raman signal produced by an analyte through surface and resonance phenomena. Analyte-shaped cavities associated with enhancing structures can be formed, which are complementary to a large number of desired analytes. Novel methods are presented for manufacturing matrices and biochips comprising enhancing structures associated with analyte-shaped cavities. The matrices and biochips of this invention can produce Raman signals that can be used for highly specific, sensitive assays of biological molecules and viruses.

### RELATED APPLICATIONS

This application claims priority to U.S. Provisional Patent Application Serial No. 60/356,254, filed Feb. 11, 2002, and is a continuation-in-part of U.S. Applications Serial Nos. 10/298,725 filed Nov. 18, 2002, which is a continuation-in-part of U.S. Ser. No. 10/294,385 filed Nov. 14, 2002, which is a continuation-in-part of U.S. Ser. No. 09/925,189 filed Aug. 8, 2001, which is a continuation-in-part of U.S. Ser. No. 09/815,909 filed Mar. 23, 2001, which is a continuation-in-part of U.S. Ser. No. 09/670,4453, which claimed priority to U.S. Provisional Patent Application Serial No. 60/156,195 filed Sep. 27, 1999, now abandoned, to U.S. Serial No. 60/156,145 filed Sep. 27, 1999, now abandoned, and to No. 60/156,471 filed Sep. 27, 1999, now abandoned. Each of the above patent applications is herein incorporated fully by reference.

### BACKGROUND OF THE INVENTION

1. Field of the Invention

This invention relates to the manufacture of particle structures for analyte detection. Specifically, the invention relates to the manufacture of particle structures having receptor molecules attached to resonance domains within the particle structures. More specifically, the invention relates to the use of particle structures having analyte-shaped cavities for the detection of analytes using Raman spectroscopy.

2. Description of Related Art

The detection and quantification of molecules or “analytes” in complex mixtures containing small amounts of analyte and large numbers and amounts of other materials is a continuing challenge. As more interest is focused upon the roles of biological molecules in physiology and disease processes, the rapid accurate detection of biological molecules such as nucleic acids and proteins is becoming more important.

I. Detection of Analytes

The detection of analyte, or “ligand” molecules is an important aspect of current biology, biotechnology, chemistry, and environmental industries. Detection of ligands can be accomplished using many different methods, including the chemical methods of chromatography, mass spectroscopy, nucleic acid hybridization and immunology. Hybridization and immunological methods rely upon the specific binding of ligands to detector, or “receptor” molecules. The basis for specificity of these methods is conferred by a receptor molecule can bind in a specific fashion to the ligand molecule, thereby creating a bound complex. Upon treating the complex under conditions that favor the removal of unbound ligand, the bound ligand can be assayed. The specificity of the binding, the completeness of separating bound and unbound ligands and receptors, and the sensitivity of the detection of the ligand confers the selectivity of the detection system.

For example, in biology and biotechnology industries, analytes such as deoxyribonucleic acid (“DNA”) and messenger ribonucleic acid (“mRNA”) are important indicators of specific genetic, physiological or pathological conditions. DNA can contain important information about the genetic makeup of an organism, and mRNA can be an important indicator of which genes are active in a specific physiological or pathological condition and what proteins may be created as a result of gene activation. Additionally, the direct detection of proteins can be important to the understanding of the physiological or pathological condition of an individual.

DNA is made of a double helix of two strands, each of which is composed of a series or “sequence” of nucleotide bases. The bases found in DNA include adenine, thymine, cytosine and guanine. One strand of the double helix has a sequence of the nucleotides that can be transcribed into mRNA, herein termed a “reading strand,” and the other strand has a sequence of bases, each of which is complementary to the base in the position corresponding in the reading strand. For every adenine in the reading strand, a thymine is present in the other strand. Similarly, for every cytosine in the reading strand, a guanine is present in the other strand. For every guanine and adenine in the reading strand, a cytosine and a thymine, respectively, is found in the other strand. Thus, when the two strands are aligned properly with respect to the other, the complementary bases of each strand can form hydrogen bonds, thereby holding the two strands in a complex, or “hybrid” according to the model of Watson and Crick (“Watson-Crick” hybridization). Thus, the two strands are considered herein to be “complementary” to each other. Ribonucleic acid has a similar structure as DNA, except that thymine is typically replaced by the base uracil. However, uracil is complementary to adenine, and thus, hybridization of RNA can occur with DNA. Because the information content of nucleic acids resides significantly in the sequence of the units that make up the nucleic acid, purely chemical methods that can detect only the presence of nucleotide bases are of limited usefulness. Thus, methods for detecting the presence of specific DNA or RNA relies upon the characterization of the sequence of bases of that nucleic acid.

A. Hybridization Detection of Nucleic Acids

Many different methods are currently in use for the detection of nucleic acids and proteins, but those methods can be time-consuming, expensive, or poorly reproducible. For example, the detection of specific nucleic acid sequences in DNA or RNA molecules can be accomplished using hybridization reactions, wherein an analyte DNA or RNA molecule is permitted to attach to a complementary sequence of DNA. A complementary DNA molecule can be attached to a supporting matrix, and the bound DNA and matrix is herein termed a “substrate.” Exposing an analyte nucleic acid to a complementary substrate DNA can result in the formation of a relatively stable hybrid. Detection of the duplex DNA hybrid is characteristically carried out using methods that can detect labeled DNA analytes. The labeling is typically performed using radioactive, spin resonance, chromogenic or other labels, which are attached to the analyte molecules. Thus, when the labeled analyte attaches to the substrate, unbound analyte can be removed and the bound, or specific, analyte can be detected and quantified.

For example, to detect a mRNA molecule having a specific sequence using current methods, naturally occurring, or “native” mRNA is typically converted to a complementary DNA (“cDNA”) molecule using an enzyme called “reverse transcriptase” under conditions that incorporate a labeled nucleotide into the cDNA. Upon binding of the labeled cDNA to the hybridization substrate, the bound ligand can be detected using a radiometric technique such as scintillation counting, fluorescence or spin resonance, depending on the type of label used.

Currently available methods for the detection of nucleic acids and proteins have undesirable characteristics. The methods are time consuming, require expensive equipment and reagents, require expert manual operations, and the reagents can be environmentally hazardous. Additionally, for assaying mRNA, the methods also can be sensitive to defects in the fidelity of reverse transcription. Unless the cDNA made during reverse transcription is exactly complementary to the mRNA, the analyte will not have the same sequence as the native mRNA, and misleading results can be obtained. The amplification of nucleic acid sequences by the polymerase chain reaction (“PCR”) has been used to increase the numbers of nucleic acid molecules (complementary DNA or “cDNA”) that can be detected. PCR requires DNA polymerase enzymes to amplify the cDNA. Some DNA polymerases can insert incorrect bases into a growing strand of newly synthesized cDNA. In addition, the recognition of ceratin cDNA by DNA polymerase and primers used for PCR can vary depending on the specific sequences of DNA in the sample to be amplified. This variation can result in non-proportional amplification of different cDNA molecules. Subsequent amplification of an strand having an incorrect sequence can result in the presence of several different cDNA sequences in the same sample. Thus, the accuracy and sensitivity of analysis of cDNA using PCR can be compromised.

Additionally, for medical diagnostic or forensic purposes, it can be very important for results of tests to be available rapidly. Commonly used methods for detection of specific nucleic acid sequences can be too slow for therapeutic or forensic uses. Thus, there is a need for rapid, accurate measurement of nucleic acid sequences.

II. Raman Spectroscopy

Raman spectroscopy involves the use of electromagnetic radiation to generate a signal in an analyte molecule. Raman spectroscopic methods have only recently been developed to the point where necessary sensitivity is possible. Raman spectroscopic methods and some ways of increasing the sensitivity of Raman spectroscopy are described herein below.

A. Raman Scattering

According to a theory of Raman scattering, when incident photons having wavelengths in the near infrared, visible or ultraviolet range illuminate a certain molecule, a photon of that incident light can be scattered by the molecule, thereby altering the vibrational state of the molecule to a higher or a lower level. The vibrational state of a molecule is characterized by a certain type of stretching, bending, or flexing of the molecular bonds. The molecule can then spontaneously return to its original vibrational state. When the molecule returns to its original vibrational state, it can emit a characteristic photon having the same wavelength as the incident photon. The photon can be emitted in any direction relative to the molecule. This phenomenon is termed “Raleigh Light Scattering.”

A molecule having an altered vibrational state can return to a vibrational state different from the original state after emission of a photon. If a molecule returns to a state different from the original state, the emitted photon can have a wavelength different from that of the incident light. This type of emission is known as “Raman Scattering” named after C. V. Raman, the discoverer of this effect. If, a molecule returns to a higher vibrational level than the original vibrational state, the energy of the emitted photon will be lower (i.e., have longer wavelength) than the wavelength of the incident photon. This type of Raman scattering is termed “Stokes-shifted Raman scattering.” Conversely, if a molecule is in a higher vibrational state, upon return to the original vibrational state, the emitted photon has a lower energy (i.e., have a shorter wavelength). This type of Raman scattering is termed “anti-Stokes-shifted Raman scattering.” Because many more molecules are in the original state than in an elevated vibrational energy state, typically the Stokes-shifted Raman scattering will predominate over the anti-Stokes-shifted Raman scattering. As a result, the typical shifts of wavelength observed in Raman spectroscopy are to longer wavelengths. Both Stokes and anti-Stokes shifts can be quantitized using a Raman spectrometer.

B. Resonance Raman Scattering

When the wavelength of the incident light is at or near the frequency of maximum absorption for that molecule, absorption of a photon can elevate both the electrical and vibrational states of the molecule. The efficiency of Raman scattering of these wavelengths can be increased by as much as about 10⁸ times the efficiency of wavelengths substantially different from the wavelength of the absorption maximum. Therefore, upon emission of the photon with return to the ground electrical state, the intensity of Raman scattering can be increased by a similar factor.

C. Surface Enhanced Raman Scattering

When Raman active molecules are excited near to certain types of metal surfaces, a significant increase in the intensity of the Raman scattering can be observed. The increased Raman scattering observed at these wavelengths is herein termed “surface enhanced Raman scattering.” The metal surfaces that exhibit the largest increase in Raman intensity comprise minute or nanoscale rough surfaces, typically coated with minute metal particles. For example, nanoscale particles such as metal colloids can increase intensity of Raman scattering to about 10⁶ times or greater, than the intensity of Raman scattering in the absence of metal particles. This effect of increased intensity of Raman scattering is termed “surface enhanced Raman scattering.”

The mechanism of surface enhanced Raman scattering is not known with certainty, but one factor can affect the enhancement. Electrons can typically exhibit a vibrational motion, termed herein “plasmon” vibration. Particles having diameters of about 1/10th the wavelength of the incident light can contribute to the effect. Incident photons can induce a field across the particles, and thereby can alter the movement of mobile electrons in the metal. As the incident light cycles through its wavelength, the induced motion of electrons can follow the light cycles, thereby creating an oscillation of the electron within the metal surface having the same frequency as the incident light. The electrons' motion can produce a mobile electrical dipole within the metal particle. When the metal particles have certain configurations, incident light can cause groups of surface electrons to oscillate in a coordinated fashion, thereby causing constructive interference of the electrical field so generated, creating an area herein termed a “resonance domain.” The enhanced electric field due to such resonance domains therefore can increase the intensity of Raman scattering and thereby can increase the intensity of the signal detected by a Raman spectrometer.

The combined effects of surface enhancement and resonance on Raman scattering is termed “surface enhanced resonance Raman scattering.” The combined effect of surface enhanced resonance Raman scattering can increase the intensity of Raman scattering by about 10¹⁴ or more. It should be noted that the above theories for enhanced Raman scattering may not be the only theories to account for the effect. Other theories may account for the increased intensity of Raman scattering under these conditions.

D. Raman Methods for Detection of Nucleic Acids and Proteins

Several methods have been used for the detection of nucleic acids and proteins. Typically, an analyte molecule can have a reporter group added to it to increase the ability of an analytical method to detect that molecule. Reporter groups can be radioactive, flourescent, spin labeled, and can be incorporated into the analyte during synthesis. For example, reporter groups can be introduced into cDNA made from mRNA by synthesizing the DNA from precursors containing the reporter groups of interest. Additionally, other types of labels, such as rhodamine or ethidium bromide can intercalate between strands of bound nucleic acids in the assay and serve as reporter groups of hybridized nucleic acid oligomers.

In addition to the above methods, several methods have been used to detect nucleic acids using Raman spectroscopy. Vo-Dinh, U.S. Pat. No. 5,814,516; Vo-Dinh, U.S. Pat. No. 5,783,389; Vo-Dinh, U.S. Pat. No. 5,721,102; Vo-Dinh, U.S. Pat. No. 5,306,403. These patents are herein incorporated fully by reference. Recently, Raman spectroscopy has been used to detect proteins. Tarcha et al., U.S. Pat. No. 5,266,498; Tarcha et al., U.S. Pat. No. 5,567,628, both incorporated herein fully by reference, provide an analyte that has been labeled using a Raman active label and an unlabeled analyte in the test mixture. The above-described methods rely upon the introduction of a Raman active label, or “reporter” group, into the analyte molecule. The reporter group is selected to provide a Raman signal that is used to detect and quantify the presence of the analyte.

By requiring reporter groups to be introduced into the analyte, additional steps and time are required. Additionally, the above methods can require extensive washing of the bound and unbound Raman labeled analytes to provide the selectivity and sensitivity of the assay. Moreover, because specific Raman labels must be provided for each type of assay system used, properties of the analytes must be determined in advance of the assay.

### SUMMARY OF THE INVENTION

Thus, one object of this invention is the development of spectroscopic methods that do not rely on labeling of analyte molecules.

Another object of this invention is the development of methods for manufacturing and the manufacture of particle structures for optical detection methods including fluorescence, SERS and SERRS.

These and other objects are met by the design and manufacture of compositions and methods useful for the direct detection of Raman and/or other signals involving electromagnetic radiation. In general, compositions useful for analyte detection of the present invention can use particle structures that are designed to enhance electromagnetic signals, including Raman signals. Particle structures may be fractal, random or ordered.

In certain embodiments of this invention, particle structures can be generated using chemical methods using linkers. Such linked particle structures can be designed and manufactured to have desired properties, including but not limited to selection of wavelengths of incident electromagnetic radiation that permit the generation of enhanced Raman signals to permit sensitive detection of a variety of analytes.

In certain embodiments of this invention, Raman and other electromagnetic signals can be detected for analytes without the need for incorporation of electromagnetically active labels into analyte molecules. Methods of these embodiments as used for Raman spectroscopic methods are herein termed “reverse Raman spectroscopy” or “RRS.” Upon binding of the analyte to an analyte-shaped cavity and removal of unbound analyte, the analyte can provide the detectable Raman signal for detection and/or quantification and/or identification.

By using Raman systems as described, the binding of native or denatured molecules having the characteristic Raman signal can be detected and thereby can be easily quantified and analyzed. Therefore, these novel methods provide substantial improvements in speed, reliability and accuracy of the detection of biologically active molecules.

In other embodiments of this invention, surfaces are created that promote the surface enhancement effect of SERS. In other embodiments, Raman enhancing surfaces are made that incorporate receptors locally at resonance domains, thereby increasing the sensitivity of Raman spectroscopic detection.

In yet other embodiments of this invention, systems for analysis of biologically significant moieties are provided, wherein a particle structure, receptor and analyte are exposed to incident electromagnetic radiation, and the Raman spectrum of the complexes are used to detect and/or quantify the amounts of analyte present.

In some embodiments, receptors can be attached to or placed near resonance domains, thereby concentrating the productive signal and increasing the sensitivity of detection of analytes.

Other embodiments include analyte-shaped cavities near resonance domains to provide for capture of an analyte and thereby providing enhanced Raman detection of the analyte.

Certain embodiments include analyte-shaped cavities near resonance domains selectively, thereby decreasing the effects of analyte-cavity complexes at other locations.

In yet further embodiments of this invention, fractal particle structures can be used to enhance a Raman signal generated in the presence of an analyte, thereby providing methods for detection of signals with increased sensitivity.

Although many of the embodiments are illustrated for Raman spectroscopic detection of analytes, principles of this invention can be used for any detection system involving resonance of electromagnetic radiation, including fluorescence methods.

### BRIEF DESCRIPTION OF THE DRAWINGS

The invention will be described with respect to the particular embodiments thereof. Other objects, features, and advantages of the invention will become apparent with reference to the specification and drawings in which:

FIG. 1 is a drawing depicting particle structures of this invention used for spectroscopy.

FIG. 2 depicts particle structures of this invention that has been subjected to photoaggregation.

FIGS. 3a-3c depict a strategy of this invention for chemically linking particles to form particle structures of this invention.

FIGS. 4a-4d depict a strategy of this invention for linking pairs of particle pairs together using linker molecules, and the manufacture of particle structures of this invention.

FIG. 5 depicts another embodiment of this invention in which the linker groups are comprised of aryl di-isonitrile groups.

FIGS. 6a-6e illustrates a photolithographic method for manufacturing particle structures of this invention.

FIGS. 7a-7f depict embodiments of this invention comprising receptors having analyte-shaped cavities. FIGS. 7a-7c depict different receptors having analyte-shaped cavities and analytes which fit into each cavity. FIG. 7d depicts an array of analyte-shaped cavities. FIG. 7e depicts an array having three different analyte-shaped cavities and enhancing structures of this invention within the matrix of the array. FIG. 7f depicts an array having three different analyte-shaped cavities with enhancing structures of this invention overlaying the array.

FIG. 8a depicts a portion of an array of this invention having receptors comprising analyte-shaped cavities thereon and having enhancing structures on the surface of the array.

FIG. 8b depicts a matrix of this invention, having defined areas, such as depicted in FIG. 8a thereon with particle structures and analyte-shaped cavities within each area.

FIGS. 8c-8g describe embodiments of this invention wherein analyte-shaped cavities are made complementary to viruses.

FIGS. 9a-9b are graphs illustrating the principle of this invention involving the use of an oligonucleotide receptor not having adenine in Raman spectroscopic detection of oligonucleic acids that contain adenine.

FIG. 9c is a graph showing the Raman spectrum of guanine.

FIGS. 10a-10c depict a methods for manufacturing nested particle structures of this invention. FIG. 10a depicts two particles having complementary oligonucleic acid sequences aligned to hold the particles in relationship with each other. FIG. 10b depicts a first-order nested particle structure of this invention. FIG. 10c depicts a second-order nested particle structure of this invention.

FIGS. 11a-11g depict methods for manufacturing biochips of this invention.

FIG. 11a depicts a substrate used for subsequent attachment of particle structures. FIG. 11b depicts a substrate as in FIG. 11a having thiol groups.

FIG. 11c depicts particles of different sizes used to manufacture particle structures of this invention.

FIG. 11d depicts a group of nested particle structures of this invention.

FIG. 11e depicts a group of chemically linked particle structures of this invention.

FIG. 11f depicts a portion of a biochip of this invention having nested particle structures as in FIG. 11d attached to a substrate.

FIG. 11g depicts a portion of a biochip of this invention having chemically linked particle structures as in FIG. 11e attached to a substrate.

FIGS. 12a-12d depict embodiments of this invention having chemically linked particle structures and/or rods.

FIG. 12a depicts two rods useful for enhancement of electromagnetic signals.

FIG. 12b depicts a rod as shown in FIG. 12a having analyte receptors.

FIG. 12c depicts a portion of a biochip of this invention having rods with analyte receptors applied to a substrate.

FIG. 12d depicts a portion of a biochip of this invention having rods with receptors and chemically linked particles structures of this invention applied to a substrate.

FIGS. 13a-13b depict alternative embodiments of this invention. FIG. 13a depicts a top view of a portion of a biochip of this invention having rods/receptors aligned end-to end and within channels inscribed in a substrate, with and without particles. FIG. 13b depicts a cross-sectional view through a portion of a biochip of this invention as described in FIG. 13a.

### DETAILED DESCRIPTION OF THE INVENTION

Definitions

The following words and terms are used herein.

The term “analyte” as used herein includes molecules, particles or other material whose presence and/or amount is to be determined. Examples of analytes include but are not limited to deoxyribonucleic acid (“DNA”), ribonucleic acid (“RNA”), amino acids, proteins, peptides, sugars, lipids, glycoproteins, cells, sub-cellular organelles, aggregations of cells, and other materials of biological interest.

The term “fractal” as used herein includes a structure comprised of elements, and having a relationship between the scale of observation and the number of elements, i.e., scaleinvariant. By way of illustration only, a continuous line is a 1-dimensional object. A plane is a two-dimensional object and a volume is a three-dimensional object. However, if a line has gaps therein, and is not a continuous line, the dimension is less than one. For example, if ½ of the line is missing from the interior of the line, then the fractal dimension is ½. Similarly, if points on a plane are missing, the fractal dimension of the plane is between one and 2. If ½ of the points on the plane are missing, the fractal dimension is 1.5. Moreover, if ½ of the points of a solid are missing, the fractal dimension is 2.5. In scale invariant structures, the structure of objects appears to be similar, regardless of the size of the area observed. Thus, fractal structures are a type of ordered structures, as distinguished from random structures, which are not ordered.

The term “fractal associate” as used herein, includes a structure of limited size, comprising at least about 100 individual particles associated together, and which demonstrates scale invariance within an area of observation limited on the lower bound by the size of the individual particles comprising the fractal associate and on the upper bound by the size of the fractal associate.

The term “fractal dimension” as used herein, means the exponent D of the following equation: N∝R^{D}, where R is the area of observation, N is the number of particles, and D is the fractal dimension. Thus in a non-fractal solid, if the radius of observation increases by 2-fold, the number of particles observed within the volume increases by 2³. However, in a corresponding fractal, if the radius of observation increases by 2-fold, the number of particles observed increases by less than 2³.

The term “fractal particle associates” as used herein includes a large number of particles arranged so that the number of particles per unit volume (the dependent variable) or per surface unit changes non-linearly with the scale of observation (the independent variable).

The term “label” as used herein includes a moiety having a physicochemical characteristic distinct from that of other moieties that permit determination of the presence and/or amount of an analyte of which the label is a part. Examples of labels include but are not limited to fluorescence, spin-resonance, radioactive moieties. Also known as reporter group.

The term “linker” as used herein includes an atom, molecule, moiety or molecular complex having two or more chemical groups capable of binding to a surface and permitting the attachment of particles together to form groups of particles. The simplest linker connects two particles. A branched linker may link together larger numbers of particles.

The term “ordered structures” as used herein includes structures that are non-random.

The term “particle structures” as used herein includes a group of individual particles that are associated with each other in such a fashion as to permit enhancement of electric fields in response to incident electromagnetic radiation. Examples of particles include metals, metal-coated polymers and fullerenes. Also included in the meaning of the term “particle structures” are films or composites comprising particles on a dielectric surface or imbedded in a dielectric material.

The term “percolation point” as used herein means a point in time on a conductive surface or medium when the surface exhibits an increase in conductance, as measured either via surface or bulk conductance in the medium. One way to measure surface or “sheet” conductance is via electric probes applied to the surface.

The term “Raman array reader” as used herein includes a device having a light source and a light detector.

The term “Raman signal” as used herein includes a Raman spectrum or portion of Raman spectrum.

The term “Raman spectral feature” as used herein includes a value obtained as a result of analysis of a Raman spectrum produced for an analyte under conditions of detection. Raman spectral features include, but are not limited to, Raman band frequency, Raman band intensity, Raman band width, a ratio of band widths, a ratio of band intensities, and/or combinations the above.

The term “Raman spectroscopy” as used herein includes a method for determining the relationship between intensity of scattered electromagnetic radiation as a function of the frequency of that electromagnetic radiation.

The term “Raman spectrum” as used herein includes the relationship between the intensity of scattered electromagnetic radiation as a function of the frequency of that radiation.

The term “random structures” as used herein includes structures that are neither ordered nor fractal. Random structures appear uniform regardless of the point and scale of observation, wherein the scale of observation encompasses at least a few particles.

The term “receptor” as used herein includes a moiety that can bind to or can retain an analyte under conditions of detection.

The term “resonance” as used herein includes an interaction with either incident, scattered and/or emitted electromagnetic radiation and a surface having electrons that can be excited by the electromagnetic radiation and increase the strength of the electric field of the electromagnetic radiation.

The term “resonance domain” as used herein includes an area within or in proximity to a particle structure in which an increase in the electric field of incident electromagnetic radiation occurs.

The term “reporter group” as used herein means label.

The term “reverse Raman spectroscopy” (“RRS”) as used herein includes an application of Raman spectroscopy in which an analyte is distinguished by the presence of a Raman spectral feature that is not found in a receptor for that analyte or in the medium in which the analysis is performed.

The term “scaling diameter” as used herein includes a relationship between particles in a nested structure, wherein there is a ratio (scaling ratio) of particle diameters that is the same, regardless of the size of the particles.

The term “surface enhanced Raman spectroscopy” (“SERS”) as used herein includes an application of Raman spectroscopy in which intensity of Raman scattering is enhanced in the presence of an enhancing surface.

The term “surface enhanced resonance Raman spectroscopy” (“SERRS”) as used herein includes an application of Raman spectroscopy in which Raman signals of an analyte are enhanced in the presence of an enhancing surface (see SERS) and when an absorption band of the analyte overlaps with the wavelength of incident electromagnetic radiation.

Embodiments of the Invention

The methods and compositions of this invention represent improvements over the existing methods for spectroscopic methods for detection and quantification of analyte molecules. In particular, the compositions and methods can be desirable for use in conjunction with infrared spectroscopy, fluorescence spectroscopy, surface plasmon resonance, Raman spectroscopy, mass spectroscopy or any other method utilizing excitation of an analyte by electromagnetic radiation.

Certain embodiments of this invention are based upon Surface Enhanced Raman Spectroscopy (“SERS”), Surface Enhanced Resonance Raman Spectroscopy (“SERRS”) and Reverse Raman Spectroscopy (“RRS”). This invention includes methods for manufacturing Raman active structures having specific analyte receptor molecules attached to those structures. The invention also includes methods for detecting analytes using Raman spectroscopy, reverse Raman spectroscopy, compositions useful for reverse Raman spectroscopy, and arrays and test kits embodying Raman spectroscopic methods.

The structures that are desirable for use according to the methods of this invention include structures of small particles in structures, herein termed particle structures, which includes as a subset, fractal associates. Particle structures can be characterized by having physical and chemical structures that enable oscillations of electrons to be in resonance with incident and outgoing electromagnetic radiation.

I. Manufacture of Particle Structures

The Raman active structures desirable for use according to this invention can include any structure in which Raman signals can be amplified. The following discussion regarding metal fractal structures is not intended to be limiting to the scope of the invention, but is for purposes of illustration only.

A. Manufacture of Metal Particles

To make metal particles for nanoscale arrays of receptors according to some embodiments of this invention, we can generally use methods known in the art. Tarcha et al., U.S. Pat. No. 5,567,628, incorporated herein fully by reference. Metal colloids can be composed of noble metals, specifically, elemental gold or silver, copper, platinum, palladium and other metals known to provide surface enhancement. In general, to make a metal colloid, a dilute solution containing the metal salt is chemically reacted with a reducing agent. Reducing agents can include ascorbate, citrate, borohydride, hydrogen gas, and the like. Chemical reduction of the metal salt can produce elemental metal in solution, which combine to form a colloidal solution containing metal particles that are relatively spherical in shape.

### EXAMPLE 1

### Manufacture of Gold Colloid and Fractal Structures

In one embodiment of this invention, a solution of gold nuclei is made by preparing a 0.01% solution of NaAuCl₄ in water under vigorous stirring. One milliliter (“ml”) of a solution of 1% sodium citrate is added. After 1 minute of mixing, 1 ml of a solution containing 0.075% NaBH₄ and 1% sodium citrate is added under vigorous stirring. The reaction is permitted to proceed for 5 minutes to prepare the gold nuclei having an average diameter of about 2 nm). The solution containing the gold nuclei can be refrigerated at 4° C. until needed. This solution can be used as is, or can be used to produce particles of larger size (e.g., up to about 50 nm diameter), by rapidly adding 30 μl of the solution containing gold nuclei and 0.4 ml of a 1% sodium citrate solution to the solution of 1% HAuCl₄.3H₂O diluted in 100 ml H₂O, under vigorous stirring. The mixture is boiled for 15 minutes and is then cooled to room temperature. During cooling, the particles in the solution can form fractal structures. The resulting colloid and/or fractal particle structures can be stored in a dark bottle.

Deposition of enhancing particles on dielectric surfaces including glass can generate films that can enhance electromagnetic signals. Such films can be as thin as about 10 nm. In particular, the distribution of electric field enhancement on the surface of such a film can be uneven. Such enhancing areas are resonance domains. Such areas can be particular useful for positioning receptors for analyte binding and detection. For films or particle structures embedded in dielectric materials, one way to manufacture enhancing structures is to treat the surface until “percolation points” appear. Methods for measuring sheet resistance and bulk resistance are well known in the art.

### EXAMPLE 2

### Manufacture of Metal Particles and Fractal Structures Using Laser Ablation

In addition to liquid phase synthesis described above, laser ablation is used to make metal particles. A piece of metal foil is placed in a chamber containing a low concentration of a noble gas such as helium, neon, argon, xenon, or krypton. Exposure to the foil to laser light or other heat source causes evaporation of the metal atoms, which, in suspension in the chamber, can spontaneously aggregate to form fractal or other particle structures as a result of random diffusion. These methods are well known in the art.

B. Manufacture of Films Containing Particles

To manufacture substrates containing metal colloidal particles of one embodiment of this invention, the colloidal metal particles can be deposited onto quartz slides as described in Examples 1 or 2. Other films can be made that incorporate random structures or non-fractal ordered structures in similar fashions.

### EXAMPLE 3

### Manufacture of Quartz Slides Containing Gold Fractal Structures

Quartz slides (2.5 cm×0.8 cm×0.1 cm) are cleaned in a mixture of HCl:HN0₃ (3:1) for several hours. The slides are then rinsed with deionized H₂O (Millipore Corporation) to a resistance of about 18 MΩ and then with CH₃OH. Slides are then immersed for 18 hours in a solution of aminopropyltrimethoxysilane diluted 1:5 in CH₃OH. The slides are then rinsed extensively with CH₃OH (spectrophotometric grade) and deionized H₂O prior to immersion into colloidal gold solution described above. The slides are then immersed in the gold colloid solution above. During this time, the gold colloid particles can deposit and can become attached to the surface of the quartz slide. After 24 hours, colloid derivatization is complete. Once attached, the binding of colloidal gold nanocomposites to the quartz surfaces is strong and is essentially irreversible. During the procedure, ultraviolet and/or visual light absorbance spectra of such derivatized slides are used to assess the quality and reproducibility of the derivatization procedure. The manufacturing process is monitored using electron microscopy to assess the density of the colloidal coating, the distribution of gold colloid particles on the surface, and the size of the gold colloid particles.

C. Aggregation of Particles to Form Particle Structures

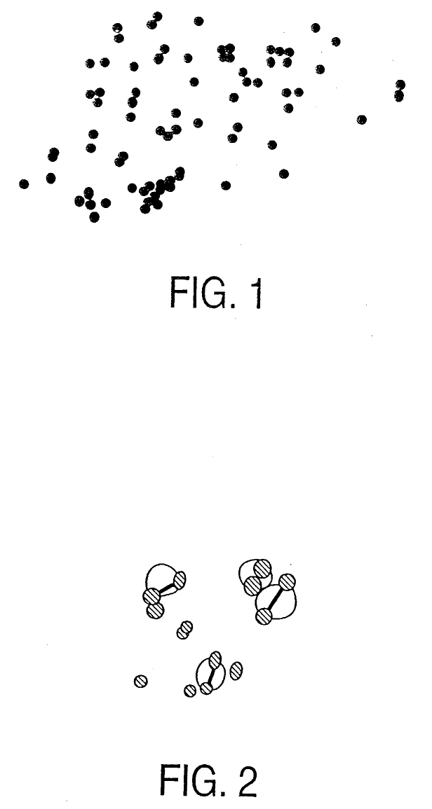

According to other embodiments of this invention, several methods can be used to form particle structures. It is known that metal colloids can be deposited onto surfaces, and when aggregated can form fractal structures having a fractal dimension of about 1.8. Safonov et al., Spectral Dependence of Selective Photomodification in Fractal Aggregates of Colloidal Particles, Physical Review Letters 80(5:1102-1105 (1998) incorporated herein fully by reference. FIG. 1 depicts a particle structure suitable for use with the methods of this invention. The particles are arranged in a scale-invariant fashion, which promotes the formation of resonance domains upon illumination by laser light.

In addition to fractal structures, ordered non-fractal structures and random structures can be generated. These different types of structures can have desirable properties for enhancing signals associated with detection of analytes using electromagnetic radiation.

To make ordered non-fractal structures, one can use, for example, chemical linkers having different lengths sequentially as described in more detail below. In addition, using linkers of the same size, one can generate ordered structures, which can be useful for certain applications.

In certain embodiments of this invention, particles can be attached together to form structures having resonance properties. In general, it can be desirable to have the particles being spheres, ellipsoids, or rods. For ellipsoidal particles, it can be desirable for the particles to have a long axis (x), another axis (y) and a third axis (z). In general, it can be desirable to have x be from about 0.05 to about 1 times the wavelength (λ) of the incident electromagnetic radiation to be used. For rods, it can be desirable for x to be less than about 4λ, alternatively, less than about 3λ, alternatively less than about 2λ, in other embodiments, less than about 1λ, and in yet other embodiments, less than about ½λ. The ends of the rods can be either flat, tapered, oblong, or have other shape that can promote resonance.

For two particle structures, it can be desirable for the particle pair to have an x dimension to be less than about 4λ, alternatively, less than about 3λ, alternatively less than about 2λ, in other embodiments, less than about 1λ, and in yet other embodiments, less than about ½.

For two-dimensional structures, pairs of particles, rods, rods plus particles together can be used. The arrangement of these elements can be randomly distributed, or can have a distribution density that is dependent upon the scale of observation in a non-linear fashion.

In other embodiments, rods can be linked together end-to end to form long structures that can provide enhanced resonance properties.

For three-dimensional structures, one can use regular nested particles, or chemical arrays of particles, associated either by chemical linkers in a fractal structure or in ordered, nested arrays.

In yet other embodiments, of third-order structures, a suspension of particles can be desirable. In certain of these embodiments, the suspended particles can have dimensions in the range of about ½λ to about 1 millimeter (mm).

Using the strategies of this invention, are searcher or developer can satisfy many needs, including, but not limited to selecting the absorbance of electromagnetic radiation by particle elements, the nature of the surface selected, the number of resonance domains, the resonance properties, the wavelengths of electromagnetic radiation showing resonance enhancement, the porosity of the particle structures, and the overall structure of the particle structures, including, but not limited to the fractal dimensions of the structure(s).

1. Photoaggregation

Photoaggregation can be used to generate particle structures that have properties which can be desirable for use in Raman spectroscopy.

Irradiation of fractal metal nanocomposites by a laser pulse with an energy above a certain threshold leads to selective photomodification, a process that can result in the formation of “dichroic holes” in the absorption spectrum near the laser wavelength (Safonov et al., Physical Review Letters 80(5):1102-1105 (1998), incorporated herein fully by reference). Selective photomodification of the geometrical structure can be observed for both silver and gold colloids, polymers doped with metal aggregates, and films produced by laser evaporation of metal targets.

One theory for the formation of selective photomodification is that the localization of optical excitations in fractal structures are prevalent in random nanocomposites. According to this theory, the localization of selective photomodification in fractals can arise because of the scale-invariant distribution of highly polarizable particles (monomers). As a result, small groups of particles having different local configurations can interact with the incident light independently of one another, and can resonate at different frequencies, generating different domains, called herein “optical modes.” According to the same theory, optical modes formed by the interactions between monomers in fractal are localized in domains that can be smaller than the optical wavelength of the incident light and smaller than the size of the clusters of particles in the colloid. The frequencies of the optical modes can span a spectral range broader than the absorption bandwidth of the monomers associated with plasmon resonance at the surface. However, other theories may account for the effects of photomodification of fractal structures, and this invention is not limited to any particular theory for operability.

Photomodification of silver fractal aggregates can occur within domains as small as about 24×24×48 nm³ (Safonov et al., Physical Review Letters 80(5:1102-1105 (1998), incorporated herein fully by reference). The energy absorbed by the fractal medium can be localized in a progressively smaller number of monomers as the laser wavelength is increased. As the energy absorbed into the resonant domains increases, the temperature at those locations can increase. At a power of 11 mJ/cm², light having a wavelength of 550 nm can produce a temperature of about 600 K (Safonov et al., Physical Review Letters 80(5):1102-1105 (1998), incorporated herein fully by reference). At this temperature, which is about one-half the melting temperature of silver, sintering of the colloids can occur (Safonov et al., Id.) incorporated herein fully by reference), thereby forming stable fractal nanocomposites.

As used in this invention, photoaggregation can be accomplished by exposing a metal colloid on a surface to pulses of incident light having a wavelengths in the range of about 400 nm to about 2000 nm. In alternative embodiments, the wavelength can be in the range of about 450 nm to about 1079 nm. The intensity of the incident light can be in the range of about 5 mJ/cm² to about 20 mJ/cm². In an alternative embodiment, the incident light can have a wavelength of 1079 nm at an intensity of 11 mJ/cm².

Fractal aggregates that are especially useful for the present invention can be made from metal particles having dimensions in the range of about 10 nm to about 100 nm in diameter, and in alternative embodiments, about 50 nm in diameter. A typical fractal structure of this invention is composed of up to about 1000 particles, and an area of the aggregate typically used for large-scale arrays can have a size of about 100 μm×100 μm.

FIG. 2 depicts a particle structure that have been photoaggregated and that are suitable for use with the methods of this invention. Local areas offusion of the metal particles can be observed (circles).

2. Chemically Directed Synthesis of Particle Structures

In certain embodiments of this invention, particle structures can be made using chemical methods. First, metal particles can be either made according to methods described above, or alternatively can be purchased from commercial suppliers (NanoGram Inc., Fremont, Calif.). Second, the particles can be joined together to form first-order structures, for example, pairs of particles. Then, the first-order structures can be joined together to form second-order structures, for example, pairs of particle pairs. Finally, third-order fractal structures can be made by joining second-order structures together.

In alternative embodiments of this invention, the formation of a fractal array of metal particles can be carried out using chemical methods. Once metal colloid particles have been manufactured, each particle can be attached to a linker molecule via a thiol or other type of suitable chemical bond. The linker molecules then can be attached to one another to link adjacent colloid particles together. The distance between the particles is a function of the total lengths of the linker molecules. It can be desired to select a stoichiometric ratio of particles to linker molecules. If too few linker molecules are used, then the array of particles will be too loose or may not form at all. Conversely, if the ratio of linker molecules to particles is too high, the array may become too tight, and may even tend to form crystalline structures, which are not random, and therefore will not tend to promote surface enhanced Raman scattering.

In general, it can be desirable to perform the linking procedure sequentially, wherein the first step comprises adding linker molecules to individual particles under conditions that do not permit cross-linking of particles together. By way of example only, such a linker can comprise an oligonucleotide having a reactive group at one end only. During this first step, the reactive end of the oligonucleotide can bind with a metal particle, thereby forming a first particle-linker species, and having a free end of the linker. The ratio of linker molecules to particles can be selected, depending on the number of linker molecules are to be attached to the particle. A second linker can be attached to another group of particles in a different reaction chamber, thereby resulting in a second linker-particle species, again with the linker having a free end.

After those reactions have progressed, the different linker-particle species can be mixed together and the linkers can attach together to form “particle pairs” joined by the linker molecules.

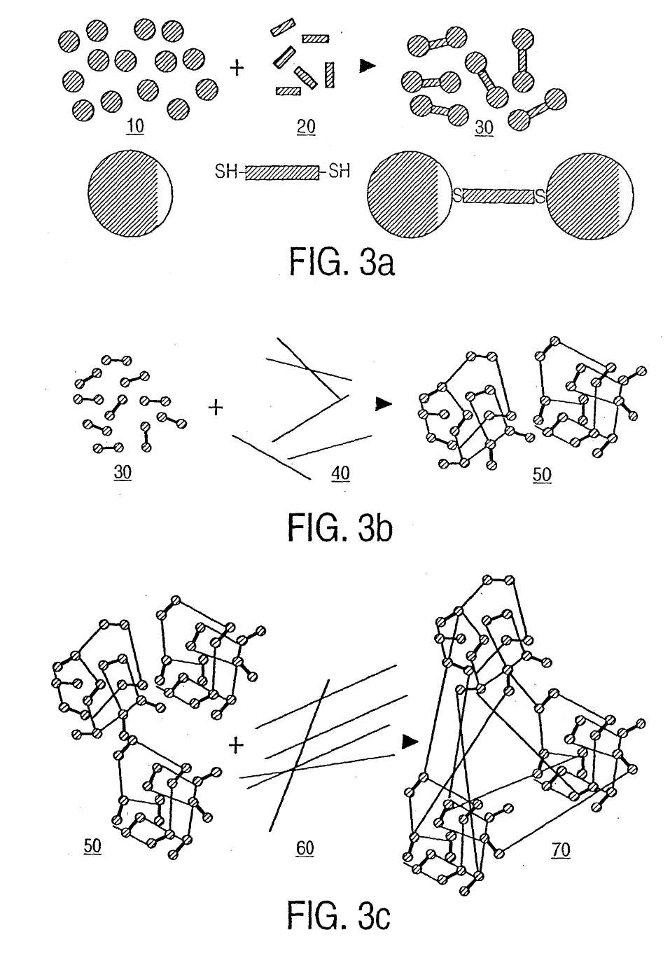

By way of example, FIGS. 3a to 3c illustrates methods for manufacturing fractal structures of this invention. In FIG. 3a, metal particles 10 are formed using methods previously described. Short linkers 20 have chemically active ends that are capable of binding to metal particles 10. For example, linker 20 has sulfhydryl (“SH”) groups at each end of the linker 20. When combined, metal particles 10 bind with the SH ends of linkers 20 to form particle pairs 30.

FIG. 3b illustrates the steps that can be used to form clusters of particle pairs. Particle pairs 30 are reacted with medium-length linkers 40 to form clusters 50.

FIG. 3c illustrates the steps that can be used to form nanoscale fractal structures of this invention. Clusters 50 are reacted with long linkers 60 to form nanoscale fractal structure 70.

In other embodiments, nucleic acids can be used as linkers, based upon the ability of DNA to form hybrids with nucleic acids comprising complementary sequences. DNA ligases or other mechanisms can be used to join the linkers together to form a complete linker between metal particles.

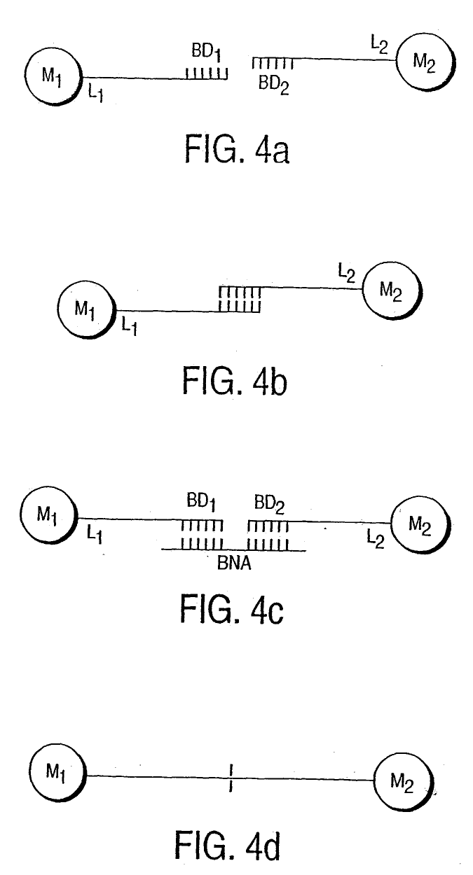

FIG. 4 depicts, in general, the linkage of metal particles to form particle pairs using linkers having binding domains. FIG. 4a depicts two metal particles (M), each having a linker molecule (L1 or L2) having a desired length, comprising inter-linker binding domains (BD1 and BD2). The inter-linker binding domains are unbound. FIG. 4b depicts the particles shown in FIG. 4a after binding of the inter-linker binding domains to form a particle pair. In embodiments in which the linkers are nucleic acids, the binding domains can have complementary sequences, such that the nucleotide residues can form stable hybrid complexes with each other, thereby linking the metal particles together as a pair. In certain embodiments, the sequence of BD1 can be poly[adenine] for example, A₁₀. The sequence of BD2 can be poly[thymidine], for example, T₁₀. Thus, A₁₀ can hybridize to T₁₀, thereby forming a stable hybrid. In other embodiments, the lengths of the binding domains can be any convenient length that permits the formation of a stable hybrid.

In other embodiments, illustrated in FIG. 4c, BD1 and BD2 can be selected to be complementary to a third nucleic acid, herein termed a “bridge nucleic acid” or (“BNA”), comprising two sequences, one complementary to BD1 and an other complementary to BD2. When the BNA is placed in contact with BD1, the portion of BNA complementary to BD1 can form a stable hybrid of the first metal particle M1 with L1 and BNA attached thereto. However, the portion of the BNA that is complementary to BD2 of L2 is free to hybridize to BD2. Upon exposure of the M1-L1-BNA complex to the M2-L2, the BD2 can bind to that portion of the BNA complementary to BD2, forming a stable particle pair.

FIG. 4d depicts an alternative particle pair in which the inter-linking molecules are attached by way of their ends. This can be accomplished, for example, by treating the particle pair shown in FIG. 4c with a DNA ligase to form a covalent bond between L1 and L2, and then by digesting away the bridge nucleic acid.

After the pairs of particles are formed, additional linkers can be attached to the particle pairs, and the process can be repeated to form “pairs of particle pairs.” Subsequently, the process can be repeated until 3 or more orders of particle structures are formed. Under these conditions, one can manufacture structures having any desired porosity. In general, the size of the nanoscale structures should have average dimensions in the range of about 20 nm to about 500 nm. In alternative embodiments, the dimensions can be in the range of about 50 nm to about 300 nm, and in other embodiments, in the range of about 100 to about 200 nm, and in yet other embodiments, about 150 nm.

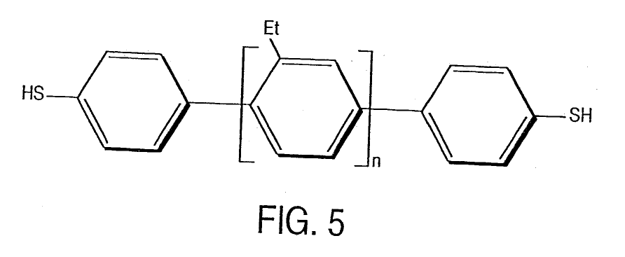

In other embodiments of this invention, the linking can be carried out using an aryl di-thiol or di-isonitrile molecules. FIG. 5 depicts the structure of a class of linkers having thiol (SH) groups at each end. Alternatively one can use any active moiety that can be used to attach the linker to the metal particle. It can be desirable to use the above types of aryl linkers with nucleic acid or other types of linker molecules. The linker can have a central area having ethylbenzene moieties, where n is a number between 1 and about 10,000.

In general, the ratio of length for each subsequent pairs of linkers can be in the range of about 2 to about 20. Alternatively, the ratios of lengths of subsequent pairs of linkers can be in the range of about 3 to about 10, and in other embodiments, about 5. In certain other embodiments, the ratio of linker lengths in successive orders can be non-constant, thus resulting in the manufacture of an ordered, non-fractal structure.

For example, for a three-order manufacturing process, it can be desirable for the ration of L1:L2:L3 to be in the range of about 1:2:4. Alternatively, the ratio can be about 1:5:25, and in yet other embodiments, the ratio can be about 1:20:400. In other embodiments, the ratio between L1 and L2 and from L2 to L3 need not be the same. Thus, in certain embodiments the ration of L1:L2:L3 can be 1:3:20, or alternatively, 1:20:40.

3. Manufacture of Suspensions of Fractal Particle Associates

In certain other embodiments of this invention, suspensions of fractal particle associates (fractal associates) can be used, for example, to provide a structure in solution that can bind or retain analytes for detection using methods of this invention. The size of fractal particle associates can be in the range of from hundreds of nanometers to mm dimensions. The fractal associates can comprise a number of particles arranged by means of chemical linkers. The number of particles per fractal associate can be as few as about 100 particles, or alternatively, thousands can be used to form a fractal associate. By increasing the number of particles in a fractal associate, the increase in the void size increases by a greater proportion.

4. Nested Fractal Structures

In another series of embodiments of this invention, nested fractal structures are provided. Nested fractal structure, for example, comprises a core of a large particle, surrounded by a “halo” of smaller particles, and each of the smaller particles is surrounded by a “halo” of even smaller particles. (See Example 6). Nested fractal structures can be especially useful for generation of essentially uniform fractal surfaces for enhanced analyte detection. It can be desirable to include large excesses of smaller particles compared to larger particles for each successive step. For example, it can be desirable to have excess of smaller particles in the range of about 10 to about 1000 times the number of larger particles. Alternatively, it can be desirable to have an excess of smaller particles of between 10 and 100 times the number of larger particles, and in other embodiments, it can be desirable to have smaller particles in excess of about 10 times the number of larger particles.

5. Lithographic Manufacture of Particle Structures

In other embodiments of this invention, particle structures can be manufactured using lithographic methods known in the semiconductor manufacturing arts. To manufacture particle structures, an image of the particle structure to be made can be made and stored in a computer memory. Each point defining the particle structure can be represented by a single location within the memory. The memory device can then direct the projection of a beam of electromagnetic radiation, electrons, or other particles locally onto a suitable surface. The beam can create site on the surface for the subsequent formation of a metal particle at desired locations.

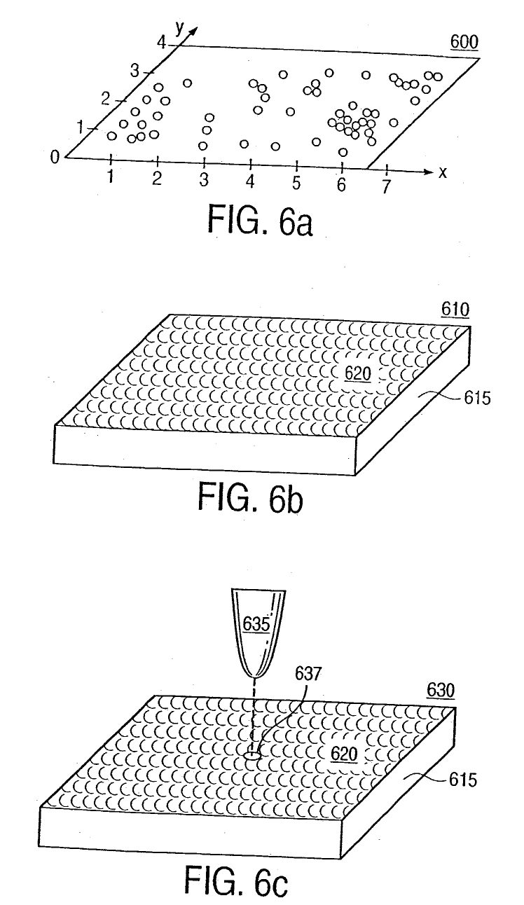

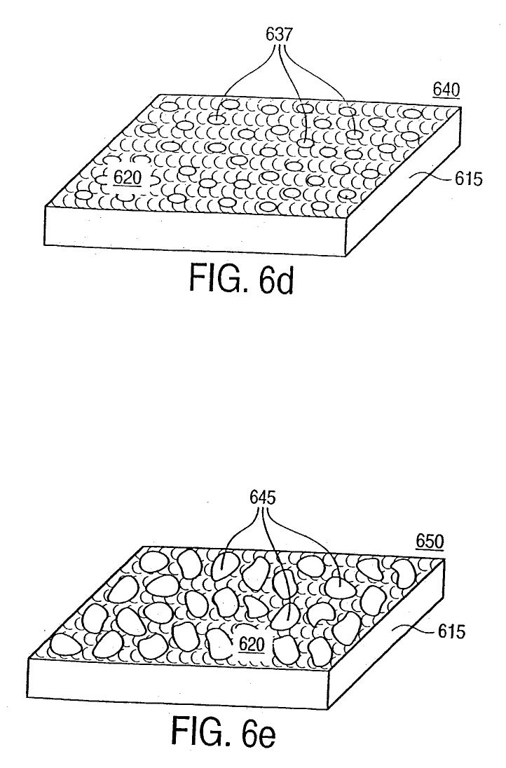

Byway of example, such a method is disclosed in Xioa et al, Hunting for the Active Sties of Surface-Enhanced Raman Scattering: A New Strategy Based on Single Silver Particles, J. Physical Chemistry B 101:632-638 (1997), incorporated herein fully by reference. FIG. 6 depicts several steps in the lithographic manufacture of a particle structure of this invention. FIG. 6a shows an image 600 of a desired distribution of nanoparticles. The image is stored in a computer memory, in which each particle is represented by a pair of reference coordinates, one x and one y for each point. FIG. 6b depicts a substrate for nanoparticle structure 610 comprising a gold substrate 615 having a film of hexadecanethiol 620 on which the nanoparticle structure is to be manufactured. FIG. 6c illustrates the placement of the tip 635 of a scanning tunneling microscope (STM) over the gold substrate 615 at a point stored in the computer memory. Electrons emitted from the tip 635 of the STM can interact with the hexadecanethiol film 620 to cause a patch 637 to form, and subsequent etching with cyanide (FIG. 6d) can expose a series of patches 637 in the surface of the underlying gold substrate 615. Thus, the pattern of particle positions stored in the computer's memory can be physically reproduced on the surface of the substrate. Subsequently, silver or other metal can be electrochemically deposited only at those locations 645 where the hexadecanethiol film 620 has been removed, thus forming the nanoparticle structure 650 (FIG. 6e).

Alternatively, traditional semiconductor masks can be used to direct the location of nanoparticle structures on substrates. Regardless of the method used, the result obtained will provide for resonance properties of the structures.

II. Manufacture of Receptor-Derivatized Particle Structures

Once the particle structures of metal particles have been manufactured, receptors can then be attached, thereby forming receptor-derivatized structures that are useful for spectroscopic detection and quantification of analytes.

A. Selection of Receptor

The receptor chosen to be attached to particle structures of this invention will depend on binding properties of the desired analyte. In general, embodiments of this invention can use analyte-shaped cavities made to suit any particular analyte of interest. Manufacture of analyte-shaped cavities in polymers are described in WO 01/61354 A1 and WO 01/61355 A1, herein incorporated fully by reference.

According to these methods, an analyte of interest can be imbedded with an unpolymerized matrix of a polymer, such as epoxy resin or other suitable material. The unpolymerized material conforms to the shape of the analyte. Then, the matrix material is polymerized. After polymerization, the analyte molecules are removed, leaving behind, analyte-shaped cavities in the matrix.

In some applications, the matrix containing analyte-shaped cavities can be divided into portions of matrix material, which on average, contain one or a few analyte-shaped cavities. In some situations, such dividing procedures can result in a certain number of portions of matrix having destroyed analyte-shaped cavities. In such situations, it can be desirable to affinity purify the matrix portions containing intact analyte-shaped cavities using, for example, an affinity column having analytes bound thereto.

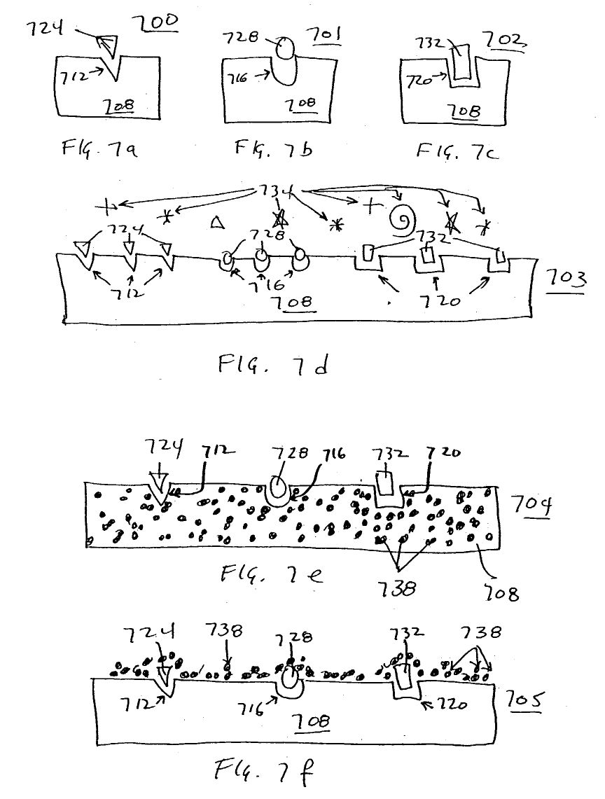

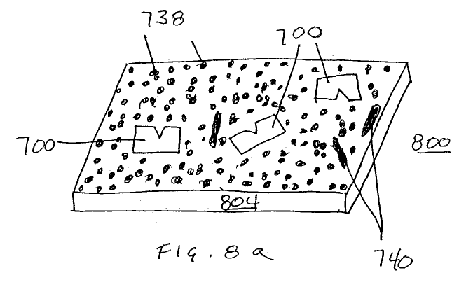

FIGS. 7a-7f depict the manufacture and use of matrices containing analyte-shaped cavities for detection of analytes. FIGS. 7a-7c depict embodiments 700, 701 and 702 each having matrix 708. FIG. 7a depicts a matrix having cavity 712, suitable for binding to analyte 724. FIG. 7b depicts a matrix having cavity 716, suitable for binding to analyte 728. FIG. 7c depicts a matrix having cavity 720, suitable for binding to analyte 732.

FIG. 7d depicts an array 703 having matrix material 708, and having cavities 712, 716 and 720 therein. These cavities, 712, 716 and 720 bind analytes 224, 728 and 732, respectively. Other compounds 734, that are not complementary to the analyte-shaped cavities 712, 716 and 720 do not bind to the array, and remain in solution above the array.

FIG. 7e depicts an embodiment of this invention 704 having matrix 708, cavities 712, 716 and 720, and analytes 724, 728, and 732 bound, respectively, thereto. The matrix 708 has enhancing structures 738 dispersed within matrix 708. Some of the enhancing structures 738 are in proximity to analytes 724, 728 and 732 to enhance Raman signals generated by the analytes.

FIG. 7f depicts an alternative embodiment of this invention 705 having matrix 708, with cavities 712, 716, and 720 and having analytes 724, 728 and 732 attached, respectively, thereto. Enhancing particle structures 738 are shown overlying the matrix 708 and analytes 724, 728 and 732.

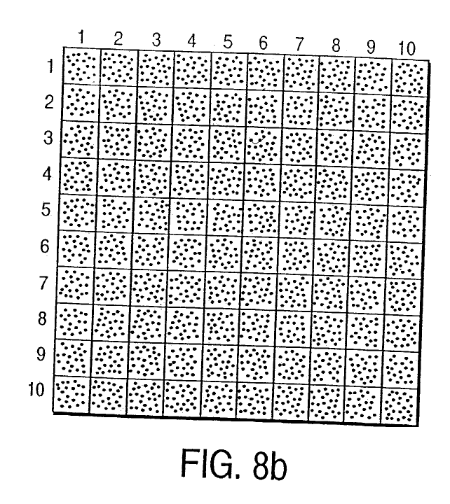

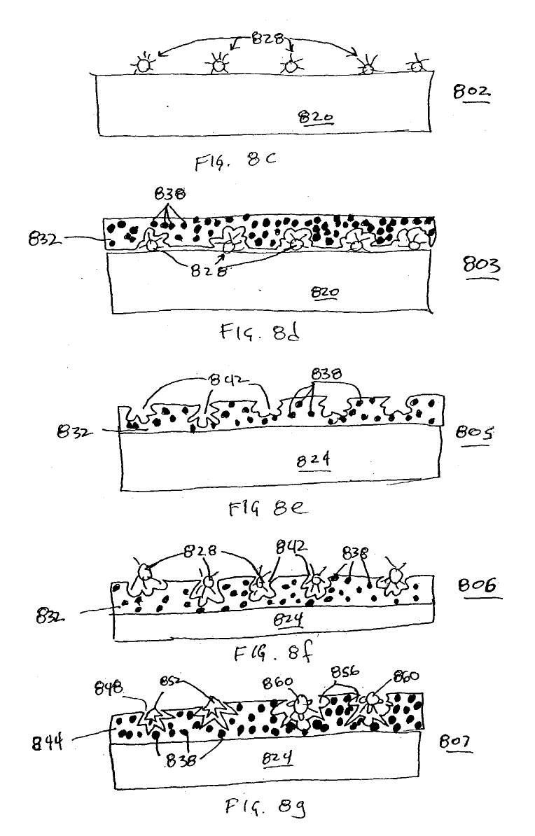

FIG. 8a depicts an embodiment of this invention 800 having matrix 804, receptors having analyte-shaped cavities 700 and enhancing structures 738 and rod-shaped enhancing structures 740.

### EXAMPLE 4

### Manufacture of an Enhancing Biochip Having Analyte Shaped Cavities

A sample of purified analyte is attached to the surface of a glass substrate using conventional methods. A mixture of unpolymerized ethylene glycol dimethylacrylate (EGDMA) solution is prepared by dissolving 2 gms of EDGMA and 0.4 gm acrylamide in 3 ml acetonitrile. We then add 1 ml of aggregated enhancing structures prepared according to methods described in Example 1 above and aggregated with NaCl (100 mM final concentration; 2 hours at room temperature). To the solution, we add a catalyst, 2, 2′-azobisisobutyronile, the solution is saturated with nitrogen for 5 minutes, is poured over the slide and allowed to polymerize under ultraviolet irradiation (370 nm for 12 hrs at 4° C.).

The resulting polymer film is then removed by peeling from the glass slide, and the film matrix, containing analyte-shaped cavities is then placed upside down on another glass slide. The film is affixed to the slide.

A sample containing an analyte used to manufacture a biochip having analyte-shaped cavities is then incubated with the surface of the slide accorind

B. Attachment of Receptors to Metal Colloid

In general, oligomers can be attached to metal surfaces via an alkanethiol covalently linked at the 5′ position of single-stranded DNA oligomers according to the methods of Herne, Characterization of DNA Probes Immobilized on Gold Surfaces, Journal American Chemical Society 119:8916-8920 (1997), incorporated herein fully by reference. The attachment can be irreversible, thereby permitting hybridization and dehybridization on the surface (Peterlinz et al., Observation of Hybridization and Dehybridization of Thiol-Derivatized DNA Using Two Color Surface Plasmon Resonance Spectroscopy. Journal American Chemical Society 119:3401-3402 (1997), incorporated herein fully by reference). However, any method can be used that results in the attachment of receptor molecules to metal surfaces and can permit the receptor to maintain the physical characteristics necessary for specific binding to ligands.

### EXAMPLE 5

### Linking of DNA to Colloidal Gold

The colloidal gold-coated quartz slides of Example 3 can then used as a matrix or substrate for the binding of DNA used for hybridization detection of analyte nucleic acids.

The gold colloid derivatized slides are placed in 1.0 M KH₂PO₄ buffer solution, pH 3.8, containing 1.0 μM thiol-derivatized DNA for a specific amount of time to achieve thiol-tethering of DNA. The surface is then passivated by exposing the DNA tethered slides to 1.0 mM mercaptohexanol (HS(CH₂)₆OH) for 1 hour. This treatment eliminates nonspecific binding of polynucleotides. Thorough rinsing with deionized water is required before analysis of hybridization.

C. Attachment of Receptors to Resonance Domains

In other embodiments of this invention, receptors can be localized to resonance domains within particle structures. Upon illumination of the particle structures, resonant domains can be heated, and that heating can cause partial melting of the metal particles. Typically, the dimensions of resonance domains are smaller than the wavelength of the incident light. The size of the resonance domains generated at certain wavelengths of incident light can be on the order of 1/25 of the wavelength of the light used in their generation. However, as the wavelength of light becomes longer, the size of the resonance domains can become smaller. Resonant domains are areas that can exhibit intense resonance, and can produce greater amplification of Raman signals than that possible in unaggregated metal or metal colloid substrates. Thus, resonance domains that are especially useful for this invention can be made using incident light, which can result in resonance domains comprising between about 4 to about 10 monomer particles.

In certain embodiments of this invention, the property of particle structures to become locally heated can be used advantageously to localize receptor molecules to those locations. To manufacture a particle structures having localization of resonance domain-specific receptors, a surface containing particle structures is prepared as above. A solution containing receptor molecules is then placed on the surface and in contact with the particle structures. Pulses of laser light are used to illuminate the surface, and at those locations where resonance domains are created, the local temperature of the reaction mixture can reach the threshold for the formation of intermolecular bonds between the particle structures and the receptor, thus attaching the receptor to the particle structures. In general, any thermosensitive chemistry for linking the receptors to the substrate can be used.

Generally, the power required to initiate receptor molecule derivatization is less than that needed for photoaggregation. It can be desirable to provide temperatures at the resonance domains in the range of about 0° C. to about 500° C., alternatively in a range of about 20° C. to about 300° C., in other embodiments, in the range of about 50° C. to about 180° C. In yet other embodiments, the temperature can be in the range of about 70° C. to about 100° C.

The temperature needed will vary with the threshold temperature required to initiate the linkage of the receptor to the metal surface. In certain embodiments, it is desirable that the temperature locally at the resonance domains remain below the temperature at which bond breakage and reversal of the bond between the receptor and the metal surface occurs.

In other embodiments of this invention, photosensitive reagents can be used to link the receptor to the particle structures at specific locations. A number of such reagents can be obtained from Pierce Products Inc., Rockford, Ill. By the use of different photochemical linking agents, one can link different types of receptors to the same substrate. For example, one can attach DNA and proteins to the same substrate.

It can be desirable to limit the attachment of receptor molecules to specific sites on a substrate. This can be accomplished by using wavelengths of light that are relatively short, for example, less than about 1000 nm, in other embodiments, below about 600 nm, in yet other embodiments, below about 400 nm. Also, laser light can be desirable in situations in which the site of attachment is to be localized to areas of high electric field. In this case, it can be desirable to use double- or triple-photon processes, in which multiple photons having long wavelengths can reach the photoreactive moiety on the receptor and particle structure to provide sufficient energy to cause a linking reaction to occur. This can occur even if the energy of a single photon is insufficient to initiate the photochemical reaction.

Once manufactured, receptor molecules localized to the resonance domains of the fractal arrays can remain at those locations during subsequent exposures to incident light.

In other embodiments of this invention, attachment of receptors at resonance domains can be performed using a scanning atomic force microscope (see Hansen et al. “A Technique for Positioning Nanoparticles Using an Atomic Force Microscope”, Nanotechnology 9:337-342 (1998), incorporated herein fully by reference), having a capillary tip and optical feedback. In these embodiments, the capillary contains derivatized receptors which can be deposited onto a surface. In the process of deposition, the surface can be illuminated by incident electromagnetic radiation produced by a laser. At resonance domains, the resonance increases the intensity of the emitted radiation and thereby provides a signal to the optical feedback device to initiate deposition of receptors at those locations, depending upon the intensity of electromagnetic radiation emitted from the surface in response to external illumination provided by the laser.

III. Design and Manufacture of Matrix Arrays

The processes described above for the derivatization of metal colloid aggregates can be extended to the manufacture of matrix arrays having a large number of different receptors. In such an array, there can be numerous individual defined areas, or “cells” that have a particular type of receptor bound to the metal colloid aggregate. The size of each cell can be on the order of about 100 μm×100 μm. Within each of these cells, a single type of receptor-fractal aggregate can be manufactured. Thus, in a matrix array of 10 cm×10 cm, there can be up to about 10⁶ different cells, each of which can have a different fractal aggregate receptor type.

FIG. 8b depicts an array comprising numerous cells or defined areas, each of which has particle structures containing a plurality of receptors bound to each defined area, and being specific for a desired analyte. The large-scale array shown is a 10×10 matrix, with individual cells positionally located within the large-scale array. Other array configurations can be desirable, and includes arrays having identifier moieties different from the receptor molecules. Identifier moieties can be used to define the position and/or the type of receptor molecule characteristic of the particular defined areas. Such identifier moieties can include nucleic acids of defined sequence, or can include identifiers produced by combinatorial chemical methods known in the art. Moreover, defined areas can be identified using colored markers.

By way of example only, a large-scale array containing fractal aggregates can be exposed to a first receptor type and a beam of highly focused incident light can selectively illuminate one or a few specific cells, thereby linking the first receptor to the substrate in only those cells in which fractal aggregates with the first receptor type is desired. Beams of highly focused laser light having the necessary dimensions can be routinely produced using of photolithography methods used in semiconductor manufacture. Subsequently, the substrate can be washed to remove unbound first receptor type, and a second receptor type can be applied to the substrate. Laser light can illuminate different cells to link the second receptor type to fractal aggregates to form fractal aggregates with the second receptor type. The process of sequential application of any desired number of different receptor types to different cells in the matrix array can be carried out using the same chemistry of linkage if desired, or different types of chemical linkage can be used. The methods above can be fully automated, so that the reproducibility of manufacture of fractal aggregates can be quite high.

A result of this process is that a matrix array containing a large number of positionally identifiable cells can be manufactured. Such arrays can be used to detect and determine sequences of DNA or mRNA, using strategies as described in, for example, U.S. Pat. No. 5,925,525, incorporated herein fully by reference.

IV. Manufacture of Arrays for Detection of Viruses

To manufacture arrays useful for detecting viruses, one can use the process described above for manufacturing arrays useful for detecting analyte molecules. Viruses may be attached to a substrate using one or more methods known in the art. For example, specific viral coat proteins may be used, or alternatively, phage display libraries may be suitable for screening large numbers of virus types in, for example, a viral panel. By attaching viral particles in a specific position, one can produce a number of different virus-shaped cavities that recognize the same viral type. Such types of multiple shaped cavities can reduce the dependence of binding affinity of a particular virus component on the overall sensitivity of detection.

Computerized analysis of Raman spectra can be used to further increase the reliability of viral detection.

FIGS. 8c-8g depict steps in the manufacture of virus-shaped cavities. FIG. 8c depicts an embodiment 802 having substrate 820 having viruses 828 attached thereto. FIG. 8d depicts the embodiment 803 having substrate 820 and viruses 828 attached thereto, and having a layer of polymer material 832 comprising enhancing structures 838 therein.

FIG. 8e depicts embodiment 805 having a analytical substrate 824 having the polymer material 832 as depicted in FIG. 8d having enhancing structures 838 therein. However, as compared to FIG. 8d, in FIG. 8e, the polymer material 832 is inverted on analytical substrate 824, so that the virus shaped cavities 842 appear on the top surface of the analytical biochip 805.

FIG. 8f depicts an embodiment 806 comprising embodiment 805 described above and having virus particles 828 bound within virus-shaped cavities 842. Enhancing structures 838 are sufficiently close to virus particles 828 to enhance Raman signals generated by virus particles 828.

FIG. 8g depicts embodiment 807 of this invention, which is an array for detection of multiple different viral species. Polymer matrix 844 has viral-shaped cavities 848 and 856. Viruses 852 and 860, bind to cavities 848 and 856, respectively. Enhancing structures 838 are sufficiently close to viruses 848 and 860 to enhance Raman signals produced by virus particles 848 and 860.

V. Detection of Analytes

Detection of analytes according to methods of this invention includes the use of a Raman reader and a matrix array. Detection can be performed using a pre-manufactured substrate having particle structures atop the substrate. The substrate can have cells or defined areas thereon, having a single type of receptor. When a sample containing an analyte is applied to such a matrix, analytes can bind to or be retained by receptors having sufficient affinity. The matrix can then be washed to remove unbound analytes, leaving only those analytes that have a sufficient affinity for the receptors to which they are bound. The matrix array can then be subjected to analysis using a reader or be performed using a light source focused upon the array, one cell at a time. Light is projected at the cell, and reflected, scattered, or re-emitted light can be collected and transmitted to the light detector. Collected light can be analyzed for Raman spectral features, and such features can be compared with Raman features derived from known moieties. Such known spectra can be imported from external databases, which can include information on biological significance of specific analytes. Analysis of information can be performed using a computer, which can be associated with a memory device for storing a program to carry out spectral analyses. Also, an output device, such as a screen display or a printer can provide information to the user. Such comparison can be the basis for determining the amount of analyte in the cell on the matrix array. Additionally, changes in the analyte due to the conditions of measurement can be determined, and any artifacts, such as non-specific binding so introduced can be discovered.

In other embodiments, detection can be performed under conditions in which resonance of electron transition in analyte molecules does not occur. According to one theory, this situation can be created when the frequency of incident light does not overlap the absorbance band of the analyte. In these situations, it can be desirable to add a suspension of particles atop the substrate and receptor analyte complexes. Enhancement of Raman signals can be sufficient to provide a highly sensitive detection.

In certain other embodiments, a combination of resonance conditions and enhancement provided by particle structures can be desirable to provide high sensitivity.

In yet other embodiments, a Raman array reader can be used to detect and quantify the amount of analyte bound to a cell of a matrix array. A Raman reader can be sued for parallel, rapid and sensitive detection of analytes by acquiring Raman spectral features of each cell of an array and comparing the spectral features with known spectral features. Thus, the existence, identity and amount of an analyte can be determined.

In some embodiments, it can be desirable to use light sources that provide different wavelengths of light simultaneously. These sources can be less expensive and if the wavelengths are sufficiently different from each other, the interference with acquiring unique Raman spectra can be minimized.

Detection of analytes according to some embodiments of this invention is advantageously carried out using native analytes. To carry out such a detection, it can be desirable to use receptor molecules that are lacking a structural feature of the analyte that is responsible for a Raman signal. Such a strategy is termed herein, “Reverse Raman Spectroscopy,” or “RRS.” In general, nucleic acids can be detected advantageously using RRS. Several examples of this strategy follow herein below

A. Detection of Analytes by Raman Spectroscopy

Detection of analytes using RRS typically involve the use of receptor molecules that are lacking moieties present in the native analyte to be detected. For example, using analyte-shaped cavities and enhancing structures of this invention, the Raman signals produced by the polymer can be determined in advance, and distinguished from the signals produced by the analyte. It can be desirable to select polymer materials for the matrices that do not contain a characteristic Raman spectral feature of the analyte to be detected.

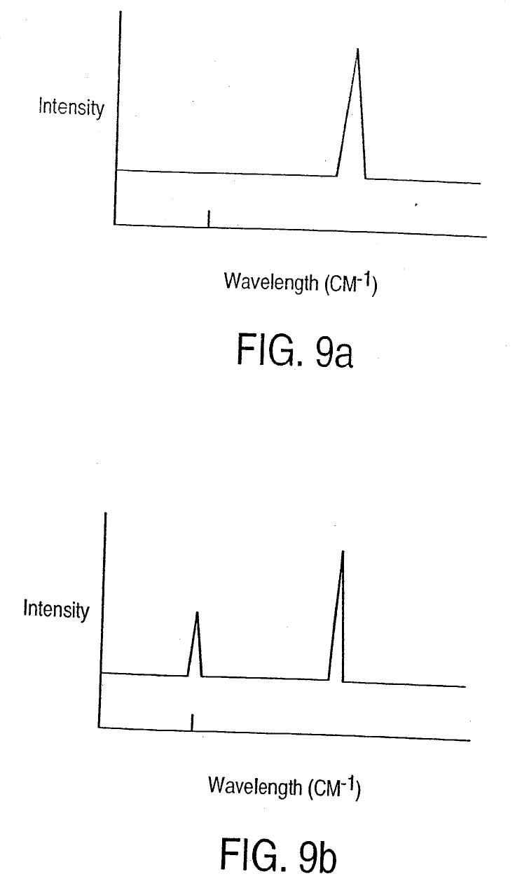

FIGS. 9a-9b are graphs illustrating the principle of use of an analyte-shaped cavity receptor not having a Raman spectroscopic feature characteristic of an analyte. FIG. 9a depicts a portion of a Raman spectrum of a polymer material having a Raman band. FIG. 9b depicts the Raman spectrum obtained upon binding of an analyte to a receptor having an analyte-shaped cavity, showing the presence of an additional Raman band.

1. Raman Spectroscopy of Analytes

Devices used to perform analyses according to the methods of this invention can include any device that can produce laser light of the wavelengths needed for analysis. For example, the T64000 Raman Spectrometer (The Ultimate Raman Spectrometer Instruments S.A. Ltd. (UK) can be used. Desirable features of a suitable instrument include the ability to position the sample compartment to adjust the sensitivity of the spectrum, provides for low frequency measurements, provides adequate spectral resolution, and a liquid nitrogen cooled charged coupled device (“CCD”) detector. The spectrometer is suitably equipped with a laser light source comprising a continuous wave, frequency doubled argon laser. Because the purine and pyrimidine ring structures of nucleotides have characteristic absorption maxima in the ultraviolet range, it can be desirable to provide laser light having emission wavelengths in the ultraviolet range. A suitable laser is the Inova 300 FReD, available from Coherent Inc., Santa Clara, Calif. Laser power for certain embodiments of this invention can be maintained at about 5 milliWatts at 257 nm, or 1 milliWatt at 244 nm, 229 nm and 238 nm.

For other applications, it can be desirable to use longer wavelengths, for example, in the range of about 830 nm. Such a light source is a continuous-wave titanium:sapphire laser. For other applications, light in the visible range can be suitable

To detect analytes in a single cell, it can be desirable to provide Raman spectroscopic measurements over areas that are sufficiently small to avoid cross-readings from adjacent cells. For matrix arrays having 100 μm×100 μm per side, it is desirable to provide a narrow, focused beam of incident light.

VI. Analysis of Data

To determine whether a cell has bound analyte, all that is needed is to compare the intensity of the characteristic Raman bands of a cell before exposure to the mixture of analytes to the intensity of the same Raman bands in the same cell after exposure to the analyte. Alternatively, for matrix arrays in which the receptors lack have a characteristic Raman spectral feature, one can use any cell prior to analyte exposure as a reference cell.

The reference cell can typically exhibit a Raman spectrum having several bands corresponding to invariant molecules. Such can be an internal standard for the comparison of cells having bound analyte. Moreover, if desired, one can incorporate into each cell, a known reference Raman label that is not present in the analyte sample. Thus, upon exposure of the cell to light under conditions of analysis, any change in light transmission or absorption that is due to non-specific Raman scattering can be evaluated in situ.

For determination of whether analyte-receptor binding occurs, a threshold increase in the intensity of a Raman spectral feature can be selected. For measurements not requiring quantification of analyte-receptor binding, this threshold can be set to a convenient, high level.

For example, about 25% of the maximal signal.

For alternative embodiments, in which the intensity of Raman signal is to be carefully assessed, it can be desirable to set the threshold to a lower value, for example, 2-5% of the maximal Raman signal.

Once the presence and/or amount of analyte is determined, subsequent operations can be carried out to provide additional information. For example, if the analysis is to determine the presence of an oligonucleotide having a desired sequence, the intensity of Raman signal from related cells can be compared. If a series of cells contains receptors having overlapping oligonucleotide sequences, as described, for example, in U.S. Pat. No. 5,925,525, incorporated herein fully by reference, then the presence of analyte in the related cells can provide information concerning the sequence and overall size of the particular analyte in question.

### EXAMPLE 6

### Manufacture of Nested Particle Associates

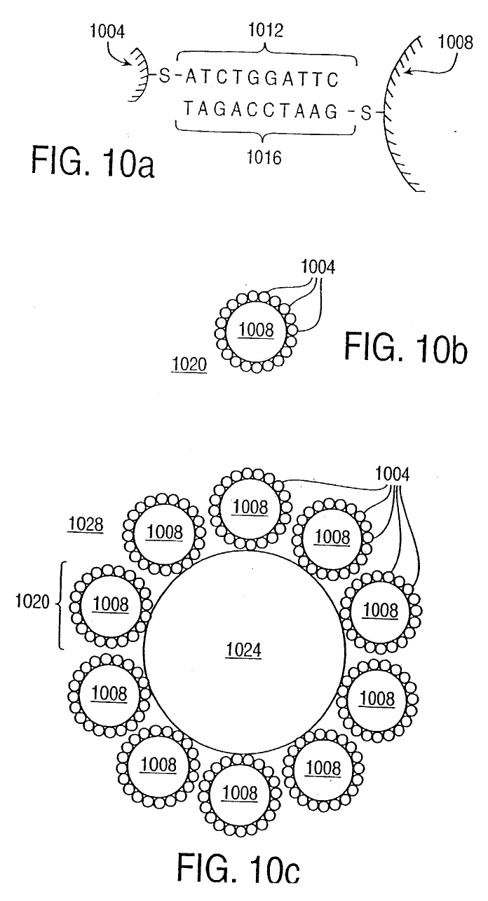

By way of example only, a nested particle associates can be made by selecting colloidal solutions of metal gold particles of uniform size, being 10 nm, 40 nm and 240 nm in diameter, respectively. FIGS. 10a-10c depict the manufacture of a nested particle structure made from such particles.

FIG. 10a depicts a 10 nm gold particle 1004 having a DNA linker 1012 attached thereto. 40 nm particle 1008 having DNA linker 1016 being complementary to DNA linker 1012 is attached to particle 1008. Mixtures of particles 1004 and 1008 are placed in solution and interact with each other DNA linkers to form a first-order nested structure 1020 as shown in FIG. 10b.

FIG. 10c depicts a second-order nested particle structure having particles 1004 and 1008 as shown in FIGS. 10a and 10b, but with the addition of a larger particle 1024 having a diameter of 240 nm, surrounded by first order nested particles 1020 to form second order nested particle 1028. Heating the mixture of first-order or second-order to a temperature less than about 100° C. and then cooling the mixture can result in better ordering of the nested particles.

### EXAMPLE 7

### Manufacture of Surfaces Having Non-Random Particle Structures

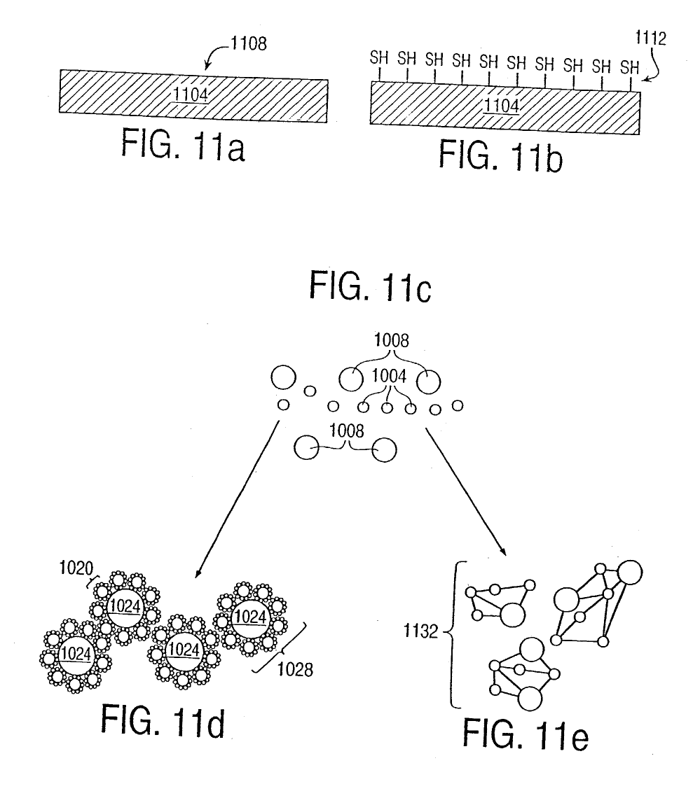

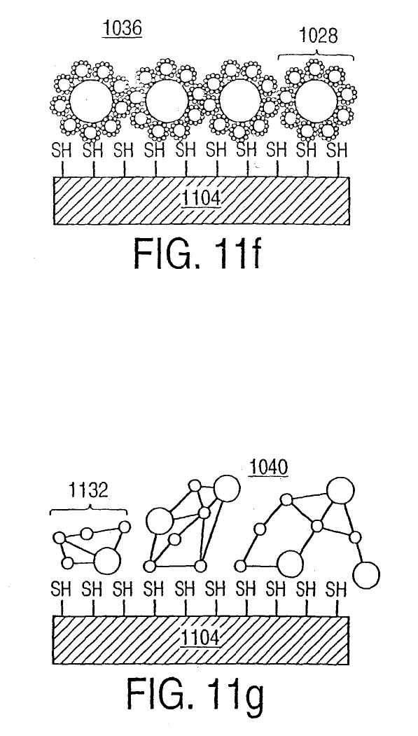

FIGS. 11a-11g depict alternative embodiments of surfaces having fractal particle structures thereon. FIG. 11a depicts a substrate 1104 having a top surface 1108. FIG. 11b depicts the surface 1008 as shown in FIG. 11a after being activated, resulting in thiol groups 1112 attached to surface 1108.

FIG. 11c depicts a plurality of particles 1004 being smaller than intermediate particles 1008. FIG. 11d depicts second-order nested particle structures 1028 made from first-order nested particles structures 1020 made from the small particles 1004, the intermediate particles 1008 and larger particles 1024.

FIG. 11e depicts chemically linked particle structures 1132 made from small particles 1004 and intermediate particles 1008.

FIG. 11f depicts an electromagnetic signal enhancer 1132 having substrate 1104 with nested particle structures 1028 thereon.

FIG. 11g depicts an alternative electromagnetic signal enhancer 1040 comprising substrate 1104 with linked particle structures 1132 as shown in FIG. 1e thereon.

### EXAMPLE 8

### Manufacture of Biochip with Analyte Receptors and Fractal Particle Structures

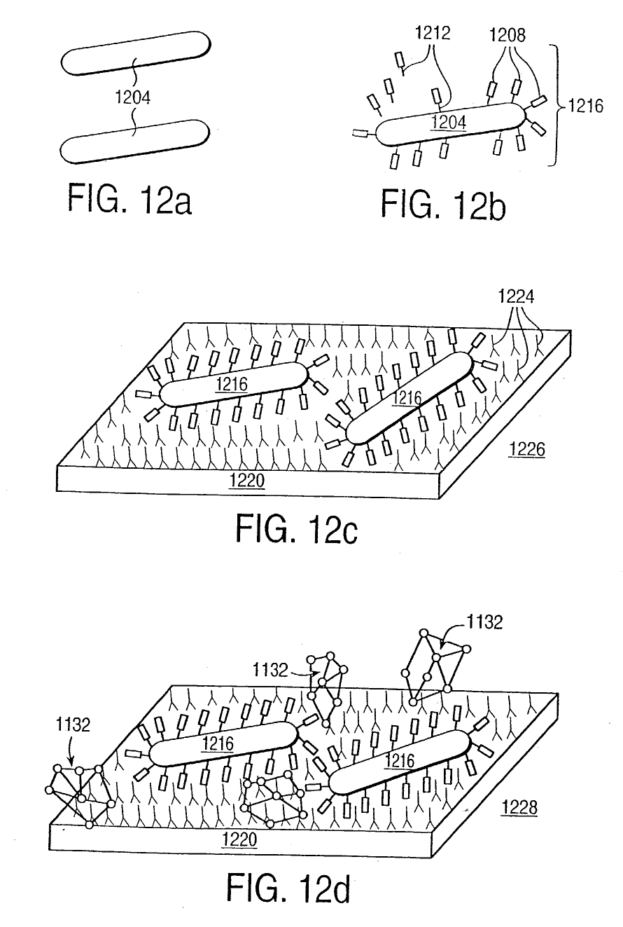

FIGS. 12a-12d depict the manufacture of a biochip having analyte receptors and enhancers. FIG. 12a depicts two rod-shaped particles 1204. FIG. 12b depicts the rod-shaped particles shown in FIG. 12a and analyte receptors 1208 with connectors 1212. Some of the analyte receptors 1208 are shown attached to rod 1024 by connectors 1212 forming receptor-rod complex 1216.

FIG. 12c depicts a biochip 1226 comprised of substrate 1220 with linkers 1224 and having receptor-rod complexes 1216 attached thereto.

FIG. 12d depicts an alternative biochip 1228, similar to biochip 1216 depicted in FIG. 12c, but further comprising linked particle structures 1132 as depicted in FIG. 11e.

### EXAMPLE 9

### Biochip Made with Non-Nested Particles

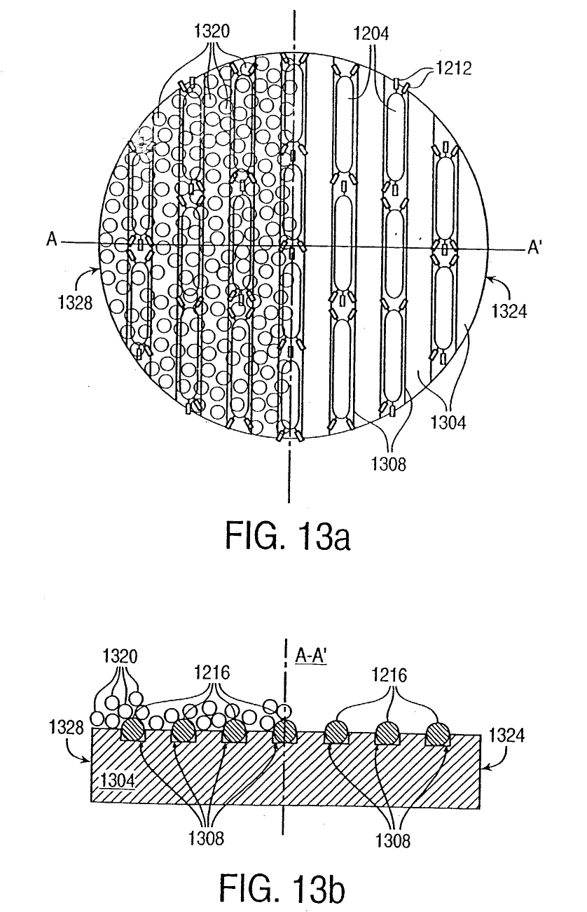

FIGS. 13a and 13b depict two views of additional embodiments 1324 of this invention having receptor-rod complexes and non-nested particles.

FIG. 13a depicts atop view of a biochip having two types of structures. On the right side of FIG. 13a, structure 1324 has linearly arranged rods 1204 having receptors 1212 attached thereto as depicted in FIG. 12b. The rods 1204 are depicted as being present within trenches 1308. Some rods 1204 are shown parallel to each other, and others are shown end-to-end, although other configurations are within the scope of this invention. The right side of FIG. 13b depicts a cross-sectional view along line A-A′ through the embodiment 1324 depicted on the right side of FIG. 13a. Trenches 1308 have receptor-rod complexes 1216 therein. The trenches 1308 can be either parallel as shown, or can be non-parallel.

The left side of FIG. 13a depicts an alternative biochip, comprising the biochip as depicted in embodiment 1324 but additionally having particles 1320 distributed over the substrate 1304 and the receptor-rod complexes. Particles 1320 can be made, for example, by laser ablation.

The left side of FIG. 13b depicts a cross-sectional view along line A-A′ of the embodiment 1328 as shown in FIG. 13a. Substrate 1304 has trenches 1308 with receptor-rod complexes 1216 therein, and having particles 1320 over the top of the substrate 1304 and receptor-rod complexes 1216.

It can be appreciated that the above descriptions and examples are intended to be illustrative of embodiments of the invention, and are not intended to limit the scope to those embodiments. Rather, persons of skill in the art can modify the disclosures and teachings of this application to arrive at embodiments that are within the scope of the invention. All of those embodiments are considered to be part of this invention.

### INDUSTRIAL APPLICABILITY

The particle structures of this invention can be used in the fields of chemistry and biotechnology for the detection of analytes in complex solutions containing many different species of molecules. Additionally, the methods of this invention can be used for the detection and quantification of analytes using spectroscopic methods, including Raman spectroscopy, fluorescence spectroscopy, immunobiology and mass spectroscopy.

We claim:

1. A device for analyte detection, comprising:
an analyte-shaped cavity; and
an enhancing structure associated with said analyte-shaped cavity.

2. The device of claim 1, further comprising an analyte molecule associated with said analyte-shaped cavity.

3. The device of claim 2, wherein said enhancing structure is sufficiently close to said analyte to enhance a Raman signal produced by said analyte.

4. The device of claim 1, wherein said analyte-shaped cavity is a virus-shaped cavity.

5. The device of claim 4, further comprising at least two virus-shaped cavities, and wherein one cavity of said at least two cavities is complementary to a first portion of said virus, and a second cavity of said at least two cavities is complementary to a second portion of said virus.

6. A biochip comprising:
a substrate
an analyte-shaped cavity; and
at least one enhancing structure associated with said analyte-shaped cavity.

7. The biochip of claim 6, comprising a plurality of analyte-shaped cavities.

8. A biochip comprising:
a substrate;
an array comprising a plurality of defined areas on said substrate, each of said defined areas having at least one analyte-shaped cavity thereon; and
at least one enhancing structure associated with said cavity.

9. The biochip of claim 8, wherein at least one of said analyte-shaped cavity is complementary to a virus.

10. The biochip of claim 6, wherein said at least analyte-shaped cavity is complementary to at least one virus.

11. A system for analyte detection, comprising:
a substrate having a plurality of defined areas thereon, each of said areas having:
at least one enhancing structure thereon; and
at least one analyte-shaped cavity; wherein each of said plurality of analyte-shaped cavities associated with each of said defined areas has a preferential analyte binding affinity different from the binding affinities of analyte-shaped cavities associated with different defined areas;
an identifier for at least one of said defined areas; and
a detector associated with a defined area.

12. The system of claim 11, further comprising a computer for storing data and for data analysis.

13. A method for analyte detection, comprising:
providing an enhancing structure;
providing an analyte-shaped cavity near said enhancing structure;
exposing said analyte to said analyte-shaped cavity to form an analyte analyte-shaped cavity complex; and
detecting a Raman spectral feature associated with said analyte receptor complex.

14. The method of claim 13, further comprising the steps of:
analyzing said spectral feature; and
comparing said spectral feature with a known reference spectral feature.

15. A method for manufacturing a matrix for analyte detection, comprising the steps of:
providing an analyte within a polymerizable matrix;
adding to said polymerizable matrix a catalyst and a plurality of enhancing structures;
permitting said polymerizable matrix to polymerize forming a polymer matrix;
removing said analytes from said polymer matrix.

16. A method of manufacturing a matrix for analyte detection, comprising the steps of:
providing a substrate having a surface and an analyte molecule attached thereto;
providing a polymerizable matrix having a plurality of enhancing structures therein;
overlaying said polymerizable matrix onto said substrate and said analyte molecule; and
permitting said polymerizable matrix to polymerize forming a polymer matrix having analyte-shaped cavities on one surface of said matrix.

17. A method for manufacturing a biochip; comprising the steps of:
providing a substrate having at least one defined area thereon;
providing a plurality of enhancing structures;
providing the polymer matrix of claim 15.

18. A method for manufacturing a biochip, comprising the steps of:
providing a substrate having at least one defined area thereon;
applying the polymer matrix of claim 16 to said at least one defined area, so that the surface of said matrix having said analyte-shaped cavities is exposed.

19. A Raman reader comprising:
a light source;
a matrix array having enhancing structures and analyte-shaped cavities thereon;
a holder for positioning said matrix array in relation to said light source;
a light detector.

20. The reader of claim 19, further comprising a computer for comparing detected light with known spectral features of known moieties.

21. The reader of claim 20, further comprising a memory device for storing a program for analyzing said spectral features.

22. The reader of claim 21, further comprising a device for acquiring information from an external database.

23. A method for detecting an analyte on a biochip, comprising the steps of:
providing a biochip having at least one defined area thereon having at least one analyte-shaped cavity thereon;
providing a Raman reader;
exposing said biochip to a solution containing an analyte;
permitting said analyte to be retained by said analyte-shaped cavity forming an analyte analyte-shaped cavity complex;
exposing said defined area to incident light; and
collecting light emitted from a defined area.
